(19) United States
(12) Patent Application Publication
(10) Pub. No.: US 2017/0233351 A1
(43) Pub. Date: Aug. 17, 2017

(54) Process for the Preparation of Olaparib and Intermediates Thereof

(71) Applicant: Apotex Inc., Toronto (CA)

(72) Inventors: Svetoslav S. Bratovanov, Ancaster (CA); Stuart P. Green, Mount Pleasant (CA)

(21) Appl. No.: 15/429,418

(22) Filed: Feb. 10, 2017

Related U.S. Application Data
(60) Provisional application No. 62/294,366, filed on Feb. 12, 2016.

(51) Int. Cl. C07D 237/32; B01J 31/24

(52) U.S. Cl. CPC C07D 237/32; B01J 31/2457

(57) ABSTRACT

The present invention provides processes for the preparation of intermediate compounds of formulas (4) and (5) useful in the preparation of Olaparib.

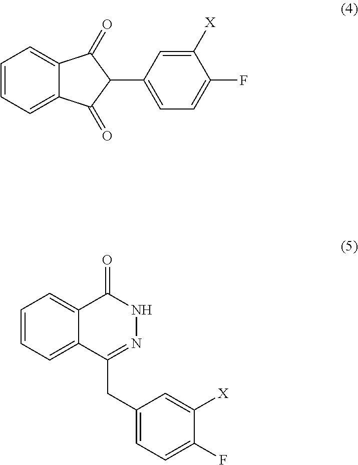

(X is chloride, bromide or iodide)

### CROSS-REFERENCE TO RELATED APPLICATION

This application claims priority to U.S. Provisional Patent Application No. 62/294,366 filed Feb. 12, 2016, the disclosure of which is hereby incorporated in its entirety by reference.

### TECHNICAL FIELD

The present invention relates to the field of synthesis of organic compounds and, in particular, to the synthesis of intermediates useful in the preparation of Olaparib.

### BACKGROUND

Olaparib (4-[(3-{([4-(cyclopropylcarbonyl)piperazin-1-yl]carbonyl}-4-fluorophenyl)methyl]phthalazin-1(2H)one), is indicated for the treatment of germline BRCA-mutated advanced ovarian cancer, and has the following structural formula (1):

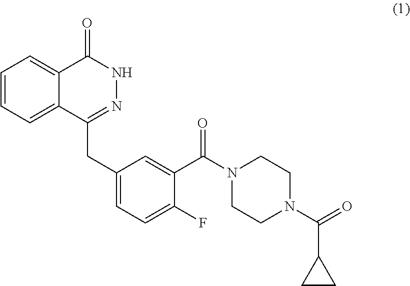 [SMILES: O=C(c1cc(Cc2n[nH]c(=O)c3ccccc23)ccc1F)N1CCN(C(=O)C2CC2)CC1]

The preparation of Olaparib is described, for example, in WO 2004/080976 A1, which discloses compounds of the formula:

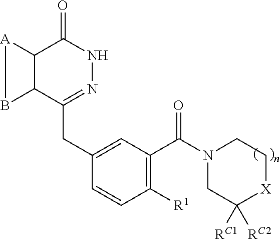

wherein A and B together represent an optionally substituted, fused aromatic ring; X can be NR^{X} or CR_{X}R^{Y}; if X is NR^{X} then n is 1 or 2 and if X=CR^{X}R^{Y} then n is 1; R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups; R^{Y} is selected from H, hydroxy, amino; or R^{X} and R^{Y} may together form a spiro-C₃₋₇ cycloalkyl or heterocyclyl group; R^{C1} and R^{C2} are both hydrogen, or when X is CR^{X}R^{Y}, R^{C1}, R_{C2}, R^{X} and R^{Y}, together with the carbon atoms to which they are attached, may form an optionally substituted fused aromatic ring; and R¹ is selected from H and halo.

In WO 2004/080976 A1, Olaparib was synthesized, as one of a number of compounds, from the intermediate compound 4-[4-fluoro-3-(piperazine-1-carbonyl)-benzyl]-2H-phthalazin-1-one (I) by the addition of cyclopropanecarbonyl chloride in dichloromethane, followed by treatment with Hünig's base (N,N-diisopropylethyl amine). This reaction is carried out with stirring at room temperature for 16 hours, with the resulting compound being purified by preparative HPLC (90% purity).

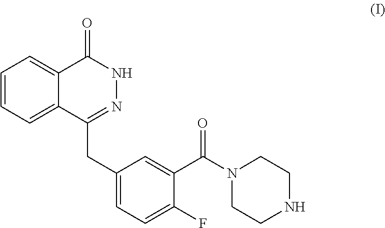 [SMILES: O=C(c1cc(Cc2n[nH]c(=O)c3ccccc23)ccc1F)N1CCNCC1]

Intermediate compound (I) was prepared by treatment of 2-fluoro-5-(4-oxo-3,4-dihydrophthalazin-1-ylmethyl)benzoic acid (II) with tent-butyl 1-piperazinecarboxylate, 2(1H-benzotriazol-1-yl)-1,1,3,3-tetramethyluronium hexafluorophosphate (HBTU) and N,N-diisopropylethyl amine in dimethylacetamide. Intermediate compound (II) was prepared by treatment of 2-carboxybenzaldehyde with sodium dimethylphosphite to form 3-oxo-1,3-dihydro-isobenzofuran-1-yl)phosphonic acid dimethyl ester, which is then converted to 2-fluoro-5-(3-oxo-3H-isobenzofuran-1-ylidenemethyl)benzonitrile, and ultimately to compound (II).

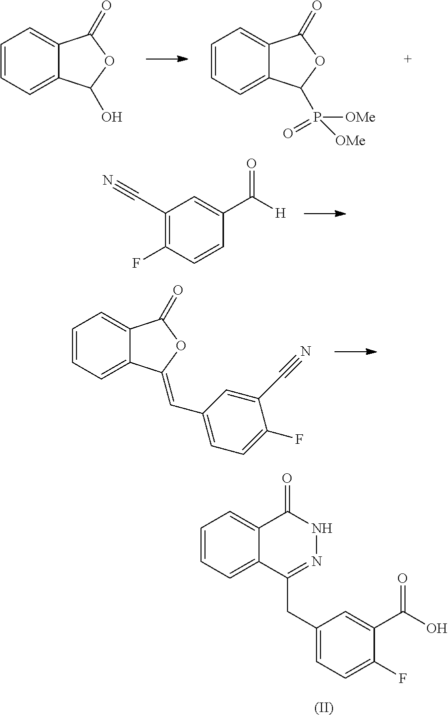

However, this process involves a number of steps and the final product is purified by HPLC. A similar process is described in Menear, K. A. et al. in J. Med. Chem. 2008, 51(20), 6581.

WO 2008/047082 A2 discloses a modified process for the preparation of Olaparib and a crystalline Form A thereof. In one aspect, a slightly more convergent process is presented wherein Olaparib is prepared by reacting compound (II) with 1-(cyclopropylcarbonyl)piperazine, or a mineral acid salt thereof, in the presence of an amide coupling agent and a base, for example, an amine. Even with this modification, the process is lengthy, requiring formation of an intermediate phosphonic acid ester.

US 2008/0280910 A1 discloses further phthalazinone derivatives and their use to inhibit the activity of the enzyme poly (ADP-ribose)polymerase-1 (PARP-1). One of the intermediates used in the preparation of these derivatives is compound (III), 4-(3-bromo-4-fluorobenzyl)phthalazin-1(2H)-one, which is known from Lescot, C. et al., J. Am. Chem. Soc. 2014, 136, 6142 to be useful in the preparation of Olaparib.

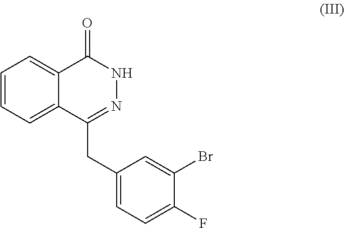 [SMILES: O=c1[nH]nc(Cc2ccc(F)c(Br)c2)c2ccccc12]

To prepare intermediate compound (III), US 2008/0280910 A1 describes a process beginning with the fusion of phthalic anhydride and 3-bromo-4-fluorophenylacetic acid in the presence of sodium acetate at 240-245° C. This type of process, which is conducted at high temperatures in the absence of solvent, is not well-suited for the industrial production of pharmaceuticals. A similar preparation is described in US 2008/0114023 A1.

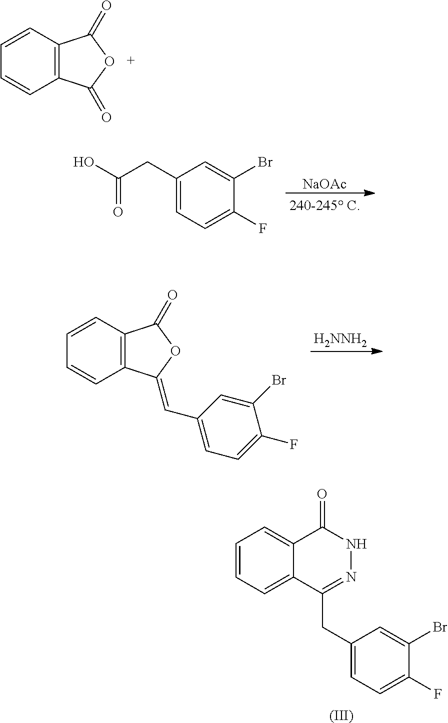

WO 2012/166983 A1 provides for compositions comprising phosphorous containing tricyclic compounds, including phthalazin-1(2H)-one derivatives. One of the intermediates used in the preparation of these derivatives is intermediate compound (III). However, like the process to prepare intermediate compound (I) in WO 2004/080976 A1, the process of WO 2012/166983 A1 involves the additional step of preparing a phosphonic acid ester intermediate.

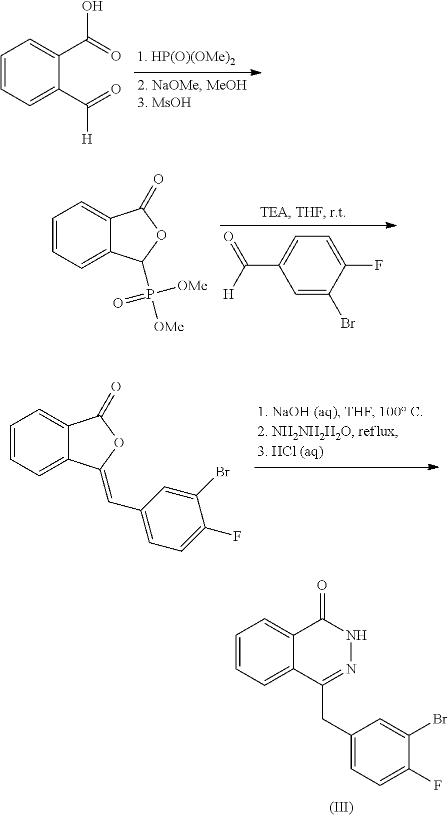 [SMILES: O=Cc1ccccc1C(=O)O ; COP(=O)(OC)C1OC(=O)c2ccccc21 ; O=Cc1ccc(F)c(Br)c1 ; O=C1O/C(=C\c2ccc(F)c(Br)c2)c2ccccc21 ; O=c1[nH]nc(Cc2ccc(F)c(Br)c2)c2ccccc12 ; "(III)"]

Kaminski, J. et al. in Przem. Chem. 1977, 56 (1), 23 discloses the synthesis of a number of 2-aryl-1 ,3-indandiones showing fungicidal properties. Included among the 2-aryl-1,3-indandiones are compounds of formula (IV), where X is Cl or Br and Y is F, Cl or Br, which are prepared through the addition of 2-(3-X-4-Y-phenyl)acetic acid to phthalic anhydride in the presence of sodium acetate at 240-250° C. followed by treatment of the intermediate with sodium methoxide in methanol. As for the process described in US 2008/0280910 A1, this type of process, which is conducted at high temperatures in the absence of solvent, is not well-suited for the industrial production of pharmaceuticals.

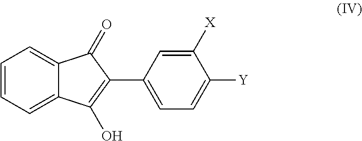

Owing to the various problems with the known processes to prepare Olaparib discussed above, there remains a need for processes for the preparation of Olaparib and the intermediates used in these preparations.

### SUMMARY

The present invention relates to synthetic processes for the preparation of compounds of Formulas (4) and (5), which are useful in the preparation of Olaparib. Using the process of the invention as described in the preferred embodiments described herein, intermediate compounds (4) and (5) are provided through industrially feasible steps in a shortened synthetic sequence. By using the new preparations of the intermediate compounds (4) and (5) described herein, a preparation of Olaparib is also provided.

Accordingly, in a first aspect of the present invention, there is provided a process for the preparation of a compound of Formula (5):

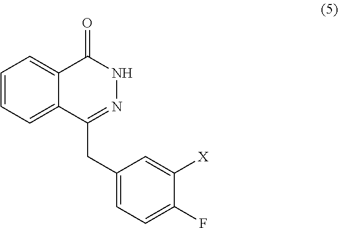

the process comprising

(i) reacting a compound of Formula (2):

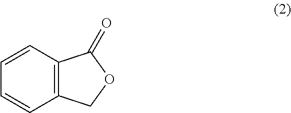

with a compound of Formula (3):

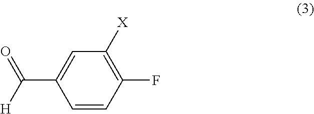

in the presence of at least about two molar equivalents of alkali metal alkoxide and at least about one molar equivalent of a compound of formula RCO₂R′, to yield a compound of Formula (4):

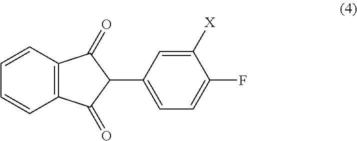

and;

(ii) reacting the compound of Formula (4) with hydrazine to produce the compound of Formula (5);

wherein

X is selected from the group consisting of chloride, bromide and iodide;

R is selected from the group consisting of hydrogen and C₁-C₄ alkyl; and

R′ is selected from the group consisting of C₁-C₄ alkyl.

In preferred embodiments of the first aspect, X in the compounds of Formulas (3), (4) and (5) is bromide; the alkali metal alkoxide is sodium methoxide, sodium ethoxide, or mixtures thereof; the compound of formula RCO₂R′ is selected from the group consisting of methyl formate, ethyl acetate, isopropyl acetate, methyl propionate, ethyl propionate, ethyl butyrate and mixtures thereof, most preferably ethyl propionate; and/or the reaction in step (ii) occurs in the presence of a base. When a base is used in the reaction of step (ii), it is preferably selected from the group consisting of metal hydroxides, metal alkoxides, carbonates, phosphates, tertiary amines and mixtures thereof. More preferably, the base is a metal hydroxide, and most preferably is sodium hydroxide.

In a second aspect of the present invention, there is provided a process for the preparation of a compound of Formula (4), the process comprising reacting a compound of Formula (2) with a compound of Formula (3) in the presence of at least about two molar equivalents of alkali metal alkoxide and at least about one molar equivalent of a compound of formula RCO₂R′, to yield the compound of Formula (4), wherein X is selected from the group consisting of chloride, bromide and iodide; R is selected from the group consisting of hydrogen and C₁-C₄ alkyl; and R′ is selected from the group consisting of C₁-C₄ alkyl.

In preferred embodiments of the second aspect, X in the compounds of Formulas (3) and (4) is bromide; the alkali metal alkoxide is sodium methoxide, sodium ethoxide or mixtures thereof; and/or the compound of formula RCO₂R′ is selected from the group consisting of methyl formate, ethyl acetate, isopropyl acetate, methyl propionate, ethyl propionate, ethyl butyrate and mixtures thereof, most preferably ethyl propionate.

In a third aspect of the present invention, there is provided a process for the preparation of a compound of Formula (5), the process comprising reacting a compound of Formula (4) with hydrazine to produce the compound of Formula (5); wherein X in the compounds of Formulas (4) and (5) is selected from the group consisting of chloride, bromide and iodide.

In a preferred embodiment of the third aspect, the reaction is conducted in the presence of a base. When a base is used in the reaction, it is preferably selected from the group consisting of metal hydroxides, metal alkoxides, carbonates, phosphates, tertiary amines and mixtures thereof. More preferably, the base is a metal hydroxide, and most preferably is sodium hydroxide.

In a fourth aspect of the present invention, there is provided a process for the preparation of Olaparib comprising: reacting a compound of Formula (2) with a compound of Formula (3) in the presence of at least about two molar equivalents of alkali metal alkoxide and at least about one molar equivalent of the compound of formula RCO₂R′, to yield a compound of Formula (4), wherein X in the compounds of Formulas (3) and (4) is selected from the group consisting of chloride, bromide and iodide; R is selected from the group consisting of hydrogen and C₁-C₄ alkyl; and R′ is selected from the group consisting of C₁-C₄ alkyl; and (ii) converting the compound of Formula (4) into Olaparib.

In a preferred embodiments of the fourth aspect, X in the compounds of Formulas (3) and (4) is bromide; the alkali metal alkoxide is sodium methoxide, sodium ethoxide, or mixtures thereof; and/or the compound of formula RCO₂R′ is selected from the group consisting of methyl formate, ethyl acetate, isopropyl acetate, methyl propionate, ethyl propionate, ethyl butyrate and mixtures thereof, most preferably ethyl propionate.

In further preferred embodiments of the fourth aspect, the compound of Formula (4) is converted into Olaparib in step (ii) by a process comprising: (a) reacting the compound of Formula (4) with hydrazine to produce a compound of Formula (5), wherein X in the compounds of Formulas (4) and (5) is selected from the group consisting of chlorine, bromide and iodide; and (b) converting the compound of Formula (5) to Olaparib.

In other preferred embodiments of the fourth aspect, X in the compounds of Formula (4) and (5) is bromide; and/or the reaction of step (ii) is conducted in the presence of a base. When a base is used in the reaction of step (ii), it is preferably selected from the group consisting of metal hydroxides, metal alkoxides, carbonates, phosphates, tertiary amines and mixtures thereof. More preferably, the base is a metal hydroxide, and most preferably is sodium hydroxide.

In yet further preferred embodiments of the fourth aspect, the conversion of the compound of Formula (5) to Olaparib in step (b) comprises reacting, in the presence of a transition metal catalyst, a ligand and carbon monoxide, the compound of Formula (5) with a compound of Formula (6) to yield Olaparib.

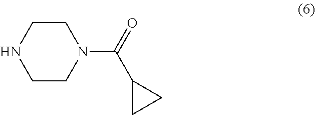 [SMILES: O=C(C1CC1)N1CCNCC1]

In preferred embodiments of the fourth aspect relating to the conversion of the compound of formula (5) to Olaparib, the transition metal catalyst is a palladium catalyst, preferably bis(dibenzylideneacetone)palladium; and/or the transition metal catalyst is bis(dibenzylideneacetone)palladium and the ligand is Xantphos (4,5-Bis(diphenylphosphino)-9,9-dimethylxanthene).

In a fifth aspect of the present invention, there is provided a process for the preparation of Olaparib comprising: (i) reacting a compound of Formula (4) with hydrazine to produce a compound of Formula (5); and (ii) converting the compound of Formula (5) to Olaparib; wherein X of compounds (4) and (5) is selected from the group consisting of chloride, bromide and iodide.

In preferred embodiments of the fifth aspect, X of the compounds of Formulas (4) and (5) is bromide; and/or the reaction of step (i) is conducted in the presence of a base. When a base is used in the reaction of step (i), it is preferably selected from the group consisting of metal hydroxides, metal alkoxides, carbonates, phosphates, tertiary amines and mixtures thereof. More preferably, the base is a metal hydroxide, and most preferably is sodium hydroxide.

In a further preferred embodiment of the fifth aspect, the conversion of the compound of Formula (5) to Olaparib in step (ii) comprises reacting, in the presence of a transition metal catalyst, a ligand and carbon monoxide, the compound of Formula (5) with a compound of Formula (6) to yield Olaparib.

In preferred embodiments of the fifth aspect relating to the conversion of the compound of Formula (5) to Olaparib, the transition metal catalyst is a palladium catalyst, preferably bis(dibenzylideneacetone)palladium; and/or the transition metal catalyst is bis(dibenzylideneacetone)palladium and the ligand is Xantphos.

### DETAILED DESCRIPTION

The present invention provides processes for the preparation of compounds of Formulas (4) and (5), which are both intermediates useful in the preparation of Olaparib. In addition to being operationally more concise, the processes to prepare the compounds of Formulas (4) and (5), when practiced according to the preferred embodiments described herein, have the advantage that the steps can be conducted in good yield using comparatively mild conditions to those used in the art, which can translate to cost savings when performed at an industrial scale. Additionally, the process of the present invention avoids the need to use 3-cyano-4-fluorobenzene acetic acid as an intermediate, presently a costly starting material, which can result in further cost savings.

As used herein, hydrazine monohydrate refers to a form of hydrazine which is 100% hydrazine monohydrate and is equivalent to 64% hydrazine: 36% water by weight. However, the use of this form of hydrazine monohydrate should not be viewed as a limitation for carrying out the processes of the invention. As would be understood by the person skilled in the art, other forms of hydrazine could be used in place of hydrazine monohydrate, for example, anhydrous hydrazine or any grade of hydrated hydrazine.

As used herein, wt % refers to weight percent and is used to express weight solute/weight solution as a percentage.

As used herein, the term “about” means close to and that variation from the exact value that follows the term within amounts that a person of skill in the art would understand to be reasonable. For example, when the term “about” is used with respect to temperature, a variation of ±5° C. is generally acceptable when carrying out the processes of the present invention.

In one embodiment of the present invention, a process is provided for the preparation of a compound of Formula (4):

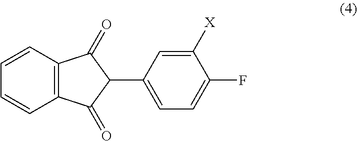

the process comprising reacting a compound of Formula (2):

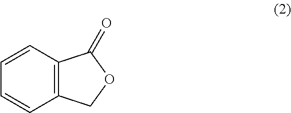

with a compound of Formula (3):

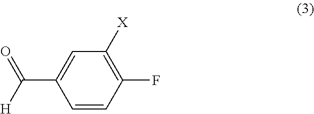

in the presence of at least about two molar equivalents of alkali metal alkoxide and at least about one molar equivalent of a compound of formula RCO₂R′, to yield a compound of Formula (4);

wherein
X is selected from the group consisting of chloride, bromide and iodide;
R is selected from the group consisting of hydrogen and C₁-C₄ alkyl; and
R′ is selected from the group consisting of C₁-C₄ alkyl.

Preferably, X in the compound of Formulas (3) and (4) is bromide.

The alkali metal alkoxide used in the preparation of the compound of Formula (4) according to the preferred embodiments of the processes of the present invention may be any appropriate alkoxide including for example, methoxide, ethoxide, t-butoxide paired with any alkali metal including for example, lithium, sodium, and potassium. Mixtures of suitable alkali metal alkoxide may also be employed. A preferred alkali metal alkoxide is sodium methoxide. The amount of alkali metal alkoxide is generally at least about 2 molar equivalents, but an excess may be used.

The compound of formula RCO₂R′ used in the preparation of the compound of Formula (4) according to the preferred embodiments of the processes of the invention is used, in combination with the alkali metal alkoxide, as a means to remove water during the course of reaction, facilitating product formation. The compound of formula RCO₂R′, wherein R and R′ are C₁-C₄-alkyl, may be any alkyl formate or ester capable of reacting with water in the presence of alkali metal alkoxide to generate the corresponding formate or acid salt. Preferred examples of the compounds of formula RCO₂R′ include methyl formate, ethyl acetate, isopropyl acetate, methyl propionate, ethyl propionate, ethyl butyrate and mixtures thereof. The amount of the compound of formula RCO₂R′ used is generally at least about 1 molar equivalent, but an excess may be used. Since the compound of formula RCO₂R′ is being used to facilitate the removal of water during the reaction, a low assay of water in the compound of formula RCO₂R′ used is desirable.

The reaction between a compound of Formula (2) and a compound of Formula (3) may be conducted in an organic solvent. While the use of an alcohol as the organic solvent is preferred, any solvent compatible with the reaction conditions may be used as an alternative solvent. Suitable alcohols may be, for example, methanol, ethanol, isopropyl alcohol, n-propanol, n-butanol, t-butyl alcohol and mixtures thereof. For convenience, the alcohol may be paired with the corresponding alkali metal alkoxide. For example, when sodium methoxide is used as the alkali metal alkoxide, methanol may be used as solvent.

The preparation of the compound of Formula (4) may be conducted at any suitable temperature from about 0° C. to about the boiling point of the reaction mixture. However, it is preferred that the reaction is conducted at between about 0° C. and about 25° C. since at higher temperatures, particularly at about 80° C. and above, the formation of undesired impurities is increased.

Depending on the specific conditions used, isolation of the compound of Formula (4) from any reaction by-products following acidification, for example, by washing the compound of Formula (4) following collection by filtration, may be necessary prior to the subsequent conversion of the compound of Formula (4) the compound of Formula (5), Olaparib.

In a second embodiment of the present invention there is provided a process for the preparation of a compound of Formula (5):

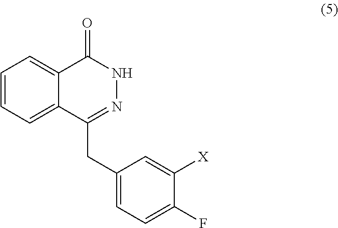

the process comprising reacting a compound of Formula (4):

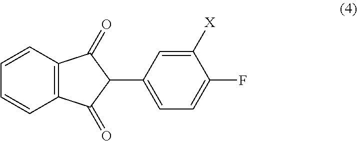

with hydrazine to produce a compound of Formula (5); wherein X is selected from the group consisting of chloride, bromide and iodide.

Preferably, X in the compounds of Formulas (4) and (5) is bromide.

When the compound of Formula (4) is obtained as a product of the reaction between a compound of Formula (2) and a compound of Formula (3), it is preferably isolated from any reaction by-products following acidification, and in particular, residual compound of formula RCO₂R′ or similar by-products, prior to the reacting the compound of Formula (4) to produce the compound of Formula (5). Preferably, the compound of Formula (5) is isolated by filtration, and optionally washing the solid material with a suitable solvent.

In the conversion of the compound of Formula (4) to the compound of Formula (5), a base may optionally be used. When a base is used in the conversion of the compound of Formula (4) to the compound of Formula (5), the base may be selected from an organic base or an inorganic base. While the base may be used catalytically, it is generally preferred that the base is used in excess to facilitate completion of the reaction. Preferred bases are selected from the group consisting of metal hydroxides (for example, sodium hydroxide, lithium hydroxide, potassium hydroxide), metal alkoxides (for example, sodium methoxide, sodium ethoxide), carbonates (for example, sodium carbonate, potassium carbonate), phosphates (for example, potassium phosphate, sodium phosphate), tertiary amines (for example, triethylamine, diisopropyl ethyl amine) and mixtures thereof. Preferred bases are metal hydroxides. Particularly preferred as the base is sodium hydroxide.

The hydrazine used may generally be provided as a hydrated form, often the 100% monohydrate form, wherein the weight % of hydrazine is 64% and the balance is water. Other aqueous solutions of hydrazine in water, for example, comprising less than 64 weight % hydrazine may be employed. However, any suitable source of hydrazine may be used. The amount of hydrazine may be from about 1 to about 20 molar equivalents. Preferably, an excess amount of 5 molar equivalents of hydrazine is used.

The conversion of a compound of Formula (4) to produce a compound of Formula (5) may be conducted in a solvent selected from a suitable aqueous or organic solvent. Preferably the solvent is selected from the group consisting of water, ethers (for example, tetrahydrofuran, methyl t-butyl ether), chlorinated hydrocarbons (for example, dichloromethane), sulfoxides (for example, dimethyl sulfoxide), amides (for example, dimethylformamide), nitriles (for example, acetonitrile), alcohols (for example, methanol) and mixtures thereof. Preferably, the solvent is water.

The conversion of the compound of Formula (5) may be conducted at any suitable temperature from about 0° C. to about the boiling point of the reaction mixture. However, it is preferred that the reaction is conducted at between about 80° C. and about 100° C.

A third embodiment of the present invention provides a process for the preparation of Olaparib wherein the compound of Formula (5), prepared by the processes of the present invention described above, is converted into Olaparib. Preferably, the conversion of the compound of Formula (5) to Olaparib comprises the addition of a compound of Formula (6) to the compound of Formula (5) in the presence of a transition metal catalyst, a ligand and carbon monoxide.

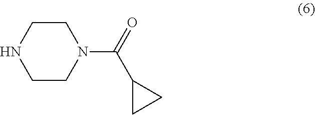 [SMILES: O=C(C1CC1)N1CCNCC1]

As one example of a suitable process, the reaction of a compound of Formula (5) and a compound of Formula (6) may be conducted according to the procedure described in Lescot, C. et al., J. Am. Chem. Soc. 2014, 136, 6142. When X in the compound of Formula (5) is bromide, the transition metal catalyst may be a suitable palladium compound such as bis(dibenzylideneacetone)palladium(0) and the ligand may be a suitable phosphorus-based ligand such as Xantphos. According to the procedure of Lescot, C. et al., a mixture of a compound of Formula (5) (1.0 equivalent, X=bromide), the compound of Formula (6) (2.0 equivalents), Pd(dba)2 (10 mol %) relative to compound (5)), Xantphos ligand (10 mol % relative to compound (5)), diisopropylethylamine (2.8 equivalents) and dioxane may be treated with carbon monoxide at 100° C. for 18 hours to generate Olaparib.

Alternatively, any other suitable process for converting a compound of Formula (5) to Olaparib can be used.

### EXAMPLES

The following examples are illustrative of some of the embodiments of the invention described herein. It will be apparent to the skilled reader that various alterations to the described processes in respect of the reactants, reagents and conditions may be made when using the processes of the present invention without departing from the scope or intent thereof.

### Example 1

### Preparation of 2-(3-bromo-4-fluorophenyl)-3-hydroxy-1H-inden-1-one (Formula (4), X=Br)

Phthalide (2.00 g, 14.92 mmol) and 3-bromo-4-fluorobenzaldehyde (3.03 g, 14.92 mmol) were dissolved in ethyl propionate (20 mL). The mixture was cooled to about 0° C. and a solution of sodium ethoxide (21 wt %) in ethanol (22.3 mL, 59.68 mmol) was added dropwise to the mixture. The mixture was allowed to warm to about 20° C. and stirred for 19 hours. The mixture was cooled to about 0° C. and quenched with water (20 mL). Following acidification to a pH of 1-2 with hydrochloric acid (10 wt %, approximately 10 mL), a dark red precipitate formed. The precipitate was collected by filtration and washed with water (30 mL). The crude solid was chromatographed (silica, ethyl acetate) to afford 2-(3-bromo-4-fluorophenyl)-3-hydroxy-1H-inden-1-one (3.05 g, 64% yield) as a dark red solid. ¹H NMR (300 MHz, CDCl₃): 8.31-8.23 (m, 1H); 8.10-7.96 (m, 1H); 7.45-7.31 (m, 5H), 97% purity by NMR. This material was used in Example 2 without further purification.

### Example 2

### Preparation of 4-(3-bromo-4-fluorobenzyl)phthalazin-1(2H)-one (Formula (5), X=Br)

A suspension of 2-(3-bromo-4-fluorophenyl)-3-hydroxy-1H-inden-1-one (1.00 g, 3.13 mmol), as obtained in Example 1, in water (20 mL) was treated with sodium hydroxide (0.51 g, 12.75 mmol) and hydrazine monohydrate (2.26 g, 45.15 mmol). The mixture was heated to 85° C. and allowed to stir for 48 hours before cooling to about 0° C. The light yellow precipitate was collected by filtration, washed with water (20 mL) and dried at room temperature in vacuo for 24 hours to afford 4-(3-bromo-4-fluorobenzyl)phthalazin-1(2H)-one (0.78 g, 75%) yield) as a light yellow solid. ¹H NMR (300 MHz, CDCl₃): 12.58 (s, 1 H); 8.25 (d, J=7.64, 1H); 7.97 (d, J=7.64, 1H); 7.91-7.81 (m, 2H); 7.67 (d, J=5.4, 2H); 7.32-7.24 (m, 2H); 3.36 (s, 2H), >98% purity by ¹H NMR.

### Example 3

### Preparation of 4-(3-bromo-4-fluorobenzyl)phthalazin-1(2H)-one (Formula (5), X=Br)

Phthalide (0.66 g, 4.92 mmol) and 3-bromo-4-fluorobenzaldehyde (1.00 g, 4.92 mmol) were dissolved in ethyl propionate (10 mL). The mixture was cooled to about 0° C. and a solution (21 wt %) of sodium ethoxide in ethanol (7.32 mL, 19.68 mmol) was added dropwise to the mixture. The mixture was heated to 80° C. and stirred for 2 hours. Following reaction completion (based on the disappearance of the phthalide by TLC), the mixture was cooled to about 0° C. Following acidification to a pH of 1-2 with hydrochloric acid (10 wt %, approximately 9.2 mL, 25.60 mmol), a dark red precipitate formed. The precipitate was collected by filtration and washed with water (20 mL). A suspension of the crude 2-(3-bromo-4-fluorophenyl)-3-hydroxy-1H-inden-1-one (1.5 g) in water (20 mL) was treated with sodium hydroxide (0.80 g, 20.00 mmol) and hydrazine monohydrate (3.43 g, 68.52 mmol). The mixture was heated to 90 ° C. and allowed to stir at 90° C. for 48 hours before cooling to about 0° C. The light yellow precipitate was filtered, washed with water (20 mL) and dried at room temperature in vacuo for 24 hours to yield 4-(3-bromo-4-fluorobenzyl)phthalazin-1(2H)-one (0.72 g, 44% yield) as a light yellow solid, approximately 97% pure by ¹H NMR.

### Example 4

### Preparation of 4-(3-bromo-4-fluorobenzyl)phthalazin-1(2H)-one (Formula (5), X=Br)

Phthalide (1.65 g, 12.30 mmol) and 3-bromo-4-fluorobenzaldehyde (2.50 g, 12.30 mmol) were dissolved in ethyl propionate (16 mL). The mixture was cooled to about 0° C. and a solution (25 wt %) of sodium methoxide in methanol (11.2 mL, 48.98 mmol) was added dropwise to the mixture. The mixture was gradually allowed to warm to room temperature over a period of 2 hours and stirred for 19 hours. The mixture was cooled to about 0° C. prior to acidification to a pH of 1-2 with hydrochloric acid (10 wt %, approximately 23 mL, 64.00 mmol), whereupon a dark red precipitate formed. The suspension was stirred at about 0° C. for 1 hour prior to collection of the precipitate by filtration. The filter cake was washed with water (20 mL). A suspension of the crude solid (3 g, wet) in water (30 mL) was treated with sodium hydroxide (2.00 g, 50.00 mmol) and hydrazine monohydrate (8.60 g, 171.79 mmol). The mixture was heated to 90° C. and allowed to stir for 28 hours prior to cooling to about 0° C. The pH of the mixture was adjusted to 9-10 with hydrochloric acid (10 wt %, approximately 103 mL, 278 mmol) and allowed to stir for 1 hour at room temperature. The red precipitate was collected by filtration and washed with water (20 mL). The crude solid (4 g, wet) was dissolved in acetone (20 mL) at room temperature. Following addition of water (20 mL), the solution was allowed to stir for 3 hours at room temperature to form a pale precipitate. The solid was collected by filtration, washed with water (20 mL) and dried in vacuo at approximately 43° C. for 24 hours to afford 4-(3-bromo-4-fluorobenzyl)phthalazin-1(2H)-one (2.97 g, 73% yield) as a light yellow solid, approximately 97% pure by 1H NMR.

### Example 5

### Preparation of 4-(3-bromo-4-fluorobenzyl)phthalazin-1(2H)-one (Formula (5), X=Br)

Phthalide (1.65 g, 12.30 mmol) and 3-bromo-4-fluorobenzaldehyde (1.65 g, 12.30 mmol) were dissolved in ethyl propionate (16 mL). The mixture was cooled to about 0° C. and a solution (21 wt %) sodium ethoxide in ethanol (18.3 mL, 49.04 mmol) was added dropwise to the mixture. The mixture was heated to room temperature and stirred for about 19 hours. The mixture was cooled to about 0° C. prior to acidification to a pH of 1-2 with hydrochloric acid (10 wt %, approximately 23 mL, 63.90 mmol), whereupon a dark red precipitate formed. The suspension was stirred at about 0° C. for 1 hour prior to collection of the precipitate by filtration and washing with water (20 mL). A suspension of the crude solid (2.1 g, wet) in water (20 mL) was treated with sodium hydroxide (0.80 g, 20.00 mmol) and hydrazine monohydrate (3.43 g, 68.52 mmol). The mixture was heated to 90° C. and allowed to stir for 48 hours prior to cooling to about 0° C. Following stirring for 1 hour at about 0° C., the light yellow precipitate was collected by filtration, washed with water (20 mL) and dried in vacuo at room temperature for 24 hours to afford 4-(3-bromo-4-fluorobenzyl)phthalazin-1(2H)-one (3.07 g 75% yield) as a light yellow solid, approximately 97% pure by ¹H NMR.

What is claimed is:

1. A process for the preparation of a compound of Formula (5): 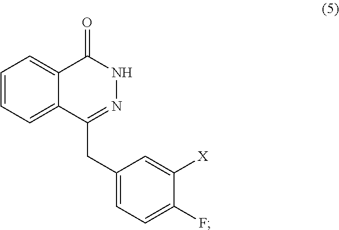
the process comprising reacting a compound of Formula (4): 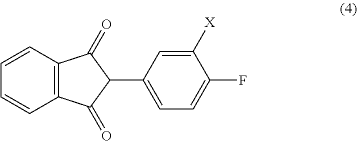
with hydrazine to produce the compound of Formula (5);
wherein
X is selected from the group consisting of chloride, bromide and iodide.

2. The process of claim 1 wherein the reaction of the compound of Formula (4) with hydrazine occurs in the presence of a metal hydroxide base.

3. A process for the preparation of Olaparib of Formula (1): 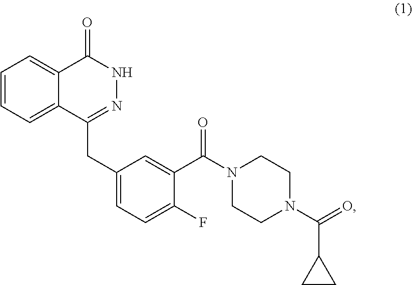 [SMILES: O=C(c1cc(Cc2n[nH]c(=O)c3ccccc23)ccc1F)N1CCN(C(=O)C2CC2)CC1]
the process comprising:
(i) reacting a compound of Formula (2): 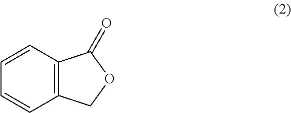
with a compound of Formula (3): 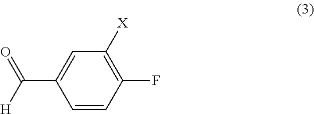
in the presence of at least about two molar equivalents of alkali metal alkoxide and at least about one molar equivalent of a compound of formula RCO₂R′, to yield a compound of Formula (4): 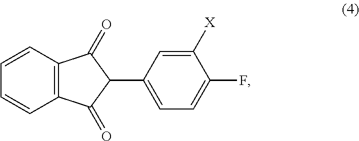
wherein
X is selected from the group consisting of chloride, bromide and iodide;
R is selected from the group consisting of hydrogen and C₁-C₄ alkyl; and
R′ is selected from the group consisting of C₁-C₄ alkyl; and
(ii) converting the compound of Formula (4) into Olaparib.

4. The process of claim 3 wherein X is bromide.

5. The process of claim 4 wherein the alkali metal alkoxide is sodium methoxide, sodium ethoxide or mixtures thereof.

6. The process of claim 5 wherein the compound of formula RCO₂R′ is selected from the group consisting of methyl formate, ethyl acetate, isopropyl acetate, methyl propionate, ethyl propionate, ethyl butyrate and mixtures thereof.

7. The process of claim 6 wherein the compound of formula RCO₂R′ is ethyl propionate.

8. The process of claim 4 wherein the conversion of the compound of Formula (4) into Olaparib in step (ii) comprises:
(a) reacting the compound of Formula (4) with hydrazine to produce a compound of Formula (5): 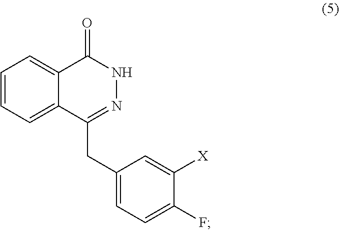
wherein X is selected from the group consisting of chloride, bromide and iodide; and
(b) converting the compound of Formula (5) to Olaparib.

9. The process of claim 7 wherein the conversion of the compound of Formula (4) into Olaparib in step (ii) comprises:
(a) reacting the compound of Formula (4) with hydrazine to produce a compound of Formula (5): 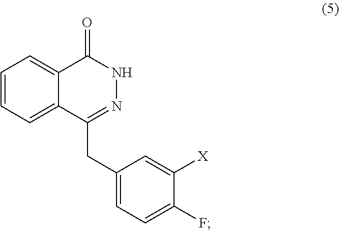
wherein X is selected from the group consisting of chloride, bromide and iodide; and
(b) converting the compound of Formula (5) to Olaparib.

10. The process of claim 8 wherein the reaction of step (a) occurs in the presence of a metal hydroxide base.

11. The process of claim 8 wherein the conversion of the compound of Formula (5) to Olaparib in step (b) comprises reacting, in the presence of a transition metal catalyst, a ligand and carbon monoxide, the compound of Formula (5) with a compound of Formula (6): 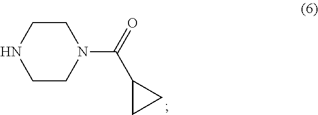 [SMILES: O=C(C1CC1)N1CCNCC1]
to yield Olaparib.

12. The process of claim 9 wherein the reaction of step (a) occurs in the presence of a metal hydroxide base and wherein the conversion of the compound of Formula (5) to Olaparib in step (b) comprises reacting, in the presence of a transition metal catalyst, a ligand and carbon monoxide, the compound of Formula (5) with a compound of Formula (6): 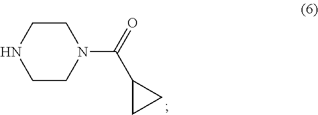 [SMILES: O=C(C1CC1)N1CCNCC1]
to yield Olaparib.

13. The process of claim 11 wherein the transition metal catalyst is bis(dibenzylideneacetone)palladium.

14. The process of claim 12 wherein the transition metal catalyst is bis(dibenzylideneacetone)palladium and the ligand is Xantphos.

15. A process for the preparation of Olaparib of Formula (1): 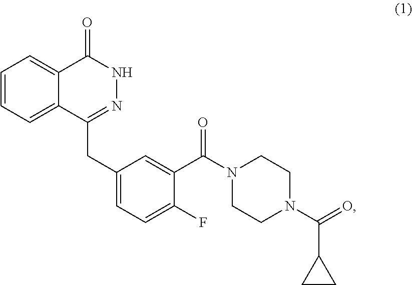 [SMILES: O=C(c1cc(Cc2n[nH]c(=O)c3ccccc23)ccc1F)N1CCN(C(=O)C2CC2)CC1]
comprising:
(i) reacting a compound of Formula (4): 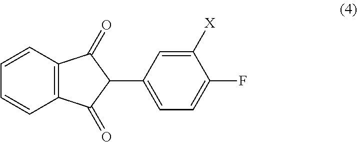
with hydrazine to produce a compound of Formula (5): 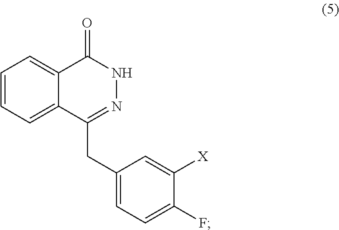
and
(ii) converting the compound of Formula (5) to Olaparib;
wherein X is selected from the group consisting of chloride, bromide and iodide.

16. The process of claim 15 wherein X is bromide.

17. The process of claim 16 wherein the reaction of step (i) occurs in the presence of a metal hydroxide base.

18. The process of claim 15 wherein the conversion of the compound of Formula (5) to Olaparib in step (ii) comprises reacting, in the presence of a transition metal catalyst, a ligand and carbon monoxide, the compound of Formula (5) with a compound of Formula (6): 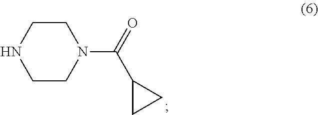 [SMILES: O=C(C1CC1)N1CCNCC1]
to yield Olaparib.

19. The process of claim 18 wherein the transition metal catalyst is bis(dibenzylideneacetone)palladium.

20. The process of claim 19 wherein the transition metal catalyst is bis(dibenzylideneacetone)palladium and the ligand is Xantphos.
